(19) United States
(12) Patent Application Publication
(10) Pub. No.: US 2011/0253801 A1
(43) Pub. Date: Oct. 20, 2011

(54) Volatile Material Dispensers

(76) Inventor: Kristian Buschmann, Chicago, IL (US)

(21) Appl. No.: 12/761,908

(22) Filed: Apr. 16, 2010

(51) Int. Cl. A61L 9/015; B23P 19/04; A61L 9/00

(52) U.S. Cl. 239/44; 239/57; 29/700

(57) ABSTRACT

A volatile material dispenser includes a housing, means for retaining a refill containing a volatile material to the housing, a channel disposed within the housing for receiving a wick disposed within the refill, and a member disposed within the channel. A first refill having a first container for holding a volatile material and having a first wick in contact with the volatile material and extending out of the first container is capable of being attached to the housing by inserting the first wick into the channel until the means for retaining retains a portion of the first refill, while a second refill having a second container for holding a volatile material and a second wick in contact with the volatile material and extending out of the second container is prevented from being attached to the housing by the member, which prevents insertion of the second wick far enough into the channel for the refill to be retained by the means for retaining, thereby preventing retention of a portion of the second refill by the means for retaining.

### CROSS REFERENCE TO RELATED APPLICATIONS

Not applicable.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

The present invention relates generally to volatile material dispensers and, more particularly, to volatile material dispensers that are adapted to be functionally coupled with only specific volatile material refills.

2. Description of the Background of the Invention

Multiple different volatile material dispensers are commercially sold and generally include a housing and a volatile material refill that is inserted into the housing. The refill generally includes a container or bottle for holding a volatile material therein. In some dispensers, the volatile material is passively emitted therefrom. In other dispensers, a diffusion element is utilized to facilitate the dispensing of the volatile material. Examples of diffusion elements include heaters, piezoelectric elements, fans, aerosol actuators, and the like. Regardless of the manner in which the volatile material is emitted, once the volatile material has been expended from the refill, the refill can typically be removed by a user and replaced with a new refill.

One type of commercial volatile material dispenser, referred to herein as a plug-in scented oil dispenser, includes a housing and a heater disposed within the housing. A refill for use with a plug-in scented oil dispenser generally includes a container portion having a bottom end and a top end, wherein the container portion terminates in a neck portion at the top end. A volatile material is disposed within the container portion and a wick is in contact with the volatile material and extends out of the refill through the neck portion. A plug or other connector generally positions and retains the wick within the neck portion. Upon insertion of the refill into the dispenser, at least a portion of the wick is disposed adjacent the heater such that volatile material that moves through the wick is volatilized by the heater.

Another feature of various volatile material dispensers and refills is that each refill has features that are unique or complementary to the particular dimensions of the housing of the dispenser for which it is sold. Still further, each type of dispenser is generally adapted to accept only a single type of refill having features unique or complementary to that dispenser. For example, plug-in scented oil dispensers sold by S. C. Johnson & Son, Inc. (“S. C. Johnson”) of Racine, Wis., generally only accept refills sold by S. C. Johnson under their Glade® brand and such refills are designed to fit only within S. C. Johnson plug-in scented oil dispensers. Similarly, plug-in scented oil dispensers sold by Reckitt Benckiser (“Reckitt”) of Berkshire, England generally only accept refills sold by Reckitt under their Air Wick® brand and such refills are designed to generally fit only within Reckitt plug-in scented oil dispensers. This is generally the case for most commercial plug-in scented oil dispensers and refills therefor.

In recent years, attempts have been made to create universal refills such that a single refill may interfit with housings of volatile material dispensers sold by multiple different companies under different brands. However, such universal refills can be unwanted, for example, by companies who intend for consumers to purchase only their refills for use with their dispensers. Users may also have developed a preference for specific refills and such universal refills can cause confusion as to the user's preferred company or brand. Further, dispensers may be designed for optimal and safe use with only specific refills.

### SUMMARY OF THE INVENTION

According to one embodiment, a volatile material dispenser includes a housing, means for retaining a refill containing a volatile material to the housing, a channel disposed within the housing for receiving a wick disposed within the refill, and a member disposed within the channel. A first refill having a first container for holding a volatile material and having a first wick in contact with the volatile material and extending out of the first container is capable of being attached to the housing by inserting the first wick into the channel until the means for retaining retains a portion of the first refill. A second refill having a second container for holding a volatile material, a second wick in contact with the volatile material and extending out of the second container, and an optional sheath disposed around the second wick is prevented from being attached to the housing by the member, which prevents insertion of the second wick or the sheath far enough into the channel for the refill to be retained by the means for retaining, thereby preventing retention of a portion of the second refill by the means for retaining.

According to another embodiment, a method of allowing only compatible refills to be retained by a volatile material dispenser includes the step of providing a volatile material dispenser that includes a housing, a channel disposed within the housing, a member disposed within the channel, and a coupling for retaining a refill. The method further includes the steps of providing a compatible refill that includes a container for holding a volatile material and a wick extending from the container and inserting the refill into the housing so that the wick extends into the channel and the coupling retains a portion of the refill. The member prevents a wick or a sheath surrounding the wick of an incompatible refill from being inserted far enough into the channel for the coupling to retain a portion of the incompatible refill assembly, thereby preventing attachment of the incompatible refill to the dispenser.

According to yet another embodiment, a volatile material dispenser includes a housing adapted to retain a compatible refill, wherein the refill includes a container for holding a volatile material and a wick extending from the container. A channel is disposed within the housing for receiving the wick and a coupling is disposed within the housing for attaching the refill to the housing. Further, an obstruction is disposed within the channel that allows the wick of the compatible refill to be inserted far enough within the channel such that the compatible refill be retained by the coupling, wherein the obstruction prevents a wick or a sheath surrounding the wick of an incompatible refill from being inserted far enough within the channel to retain the incompatible refill by means of the coupling.

Other aspects and advantages of the present invention will become apparent upon consideration of the following detailed description.

### BRIEF DESCRIPTION OF THE DRAWINGS

FIG. 1 is a top isometric view of a volatile material dispenser according to one embodiment;

FIG. 2 is a cross-sectional view taken generally along lines 2-2 of FIG. 1;

FIG. 3 is the cross-sectional view of FIG. 2 including a compatible volatile material refill functionally coupled thereto;

FIG. 4 is the cross-sectional view of FIG. 2 shown with an incompatible volatile material refill;

FIG. 5 is an isometric view of a volatile material dispenser according to another embodiment;

FIG. 6 is a cross-sectional view taken generally along lines 6-6 of FIG. 5 and including a compatible volatile material refill functionally coupled thereto;

FIG. 7 is a cross-sectional view similar to FIG. 6 shown with an incompatible volatile material refill; and

FIGS. 8-10 are top isometric views of further embodiments of volatile dispensers.

Other aspects and advantages of the present invention will become apparent upon consideration of the following detailed description, wherein similar structures have like or similar reference numerals.

### DETAILED DESCRIPTION

The present disclosure is directed to volatile material dispensers for use with only specific compatible refills. While the present disclosure may be embodied in many different forms, several specific embodiments are discussed herein with the understanding that the present disclosure is to be considered only as an exemplification of the principles of the disclosure, and it is not intended to limit the disclosure to the embodiments illustrated.

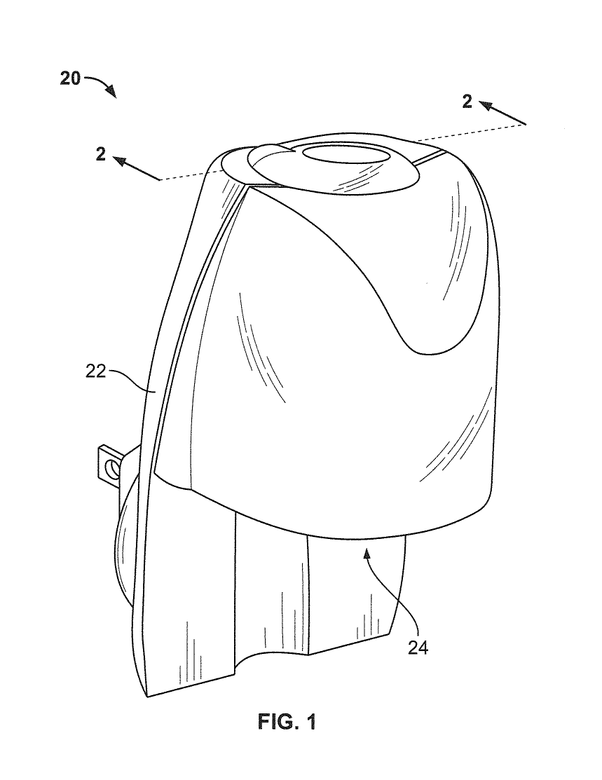

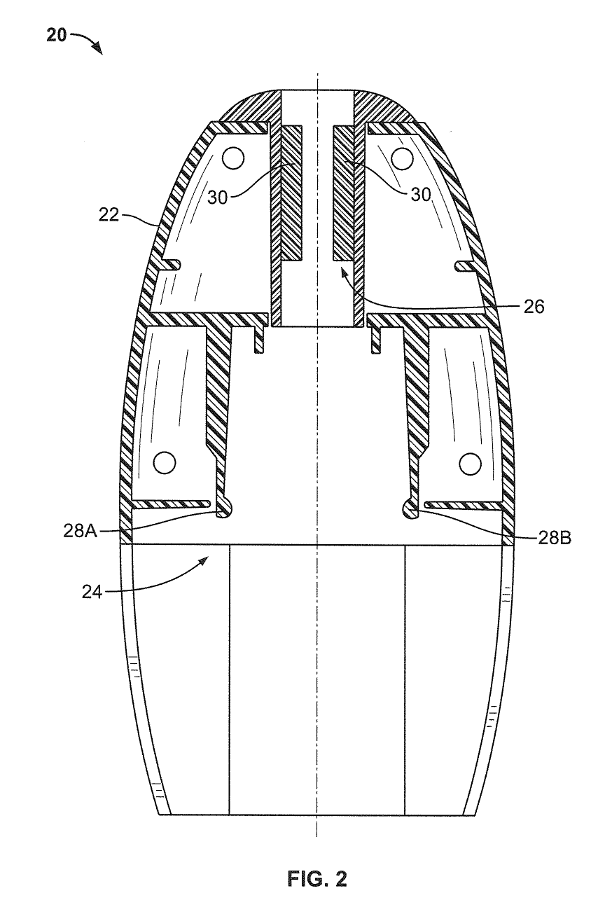

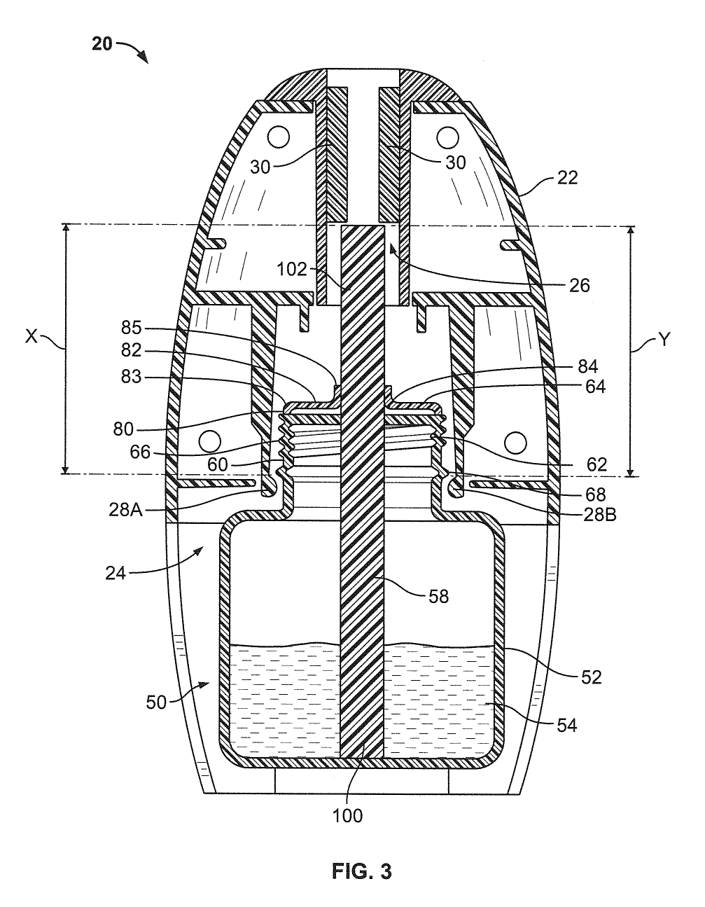

Referring now to FIGS. 1 and 2, a dispenser 20 includes a housing 22 having a cavity 24 for accepting a volatile material refill (shown in FIG. 3, for example) and an optional diffusion element disposed within the housing. The diffusion element may be a heater, a fan, a piezoelectric element, or any other diffusion element known in the art. The housing 22 further includes a channel 26 for receiving a wick disposed within the refill and resilient latches 28A, 28B that extend downwardly within the cavity 24 of the dispenser 20 to grasp a portion of the refill to attach the refill to the housing, as will be discussed in detail hereinafter.

As seen in FIG. 2, the dispenser 20 also includes a member or obstruction 30 disposed within the channel 26. The obstruction 30 is configured to prevent incompatible refills from being attached to and retained within the dispenser 20, as will be described in detail hereinafter. FIG. 2 illustrates one example of the obstruction 30 that includes a plurality of protruding members disposed around a periphery of the channel 26. In other embodiments, the obstruction 30 can be modified without departing from the spirit of the present invention, for example, the obstruction 30 may include fewer or additional protrusions that are either integral with the housing or otherwise substantially fixedly attached within the channel 26 or any other portion of the housing to prevent incompatible refills from being attached to the dispenser 20. In one embodiment, the obstruction 30 may include portions, such as, heating elements, of the optional diffusion element mentioned above.

Referring now to FIG. 3, a refill 50 compatible with and sold for use with the dispenser 20 includes a bottle or container 52 with a volatile material 54 disposed therein, wherein a wick 58 is in contact with the volatile material 54 and extends out of the container 52. The container 52 includes a neck 60 that extends from an upper portion of the container 52 and includes an opening 62 that allows access to the volatile material 54. A plug assembly 64 is disposed within the opening 62 of the neck 60 of the container 52 to hold the wick 58 within the container 52. Annular threading 66 is disposed around the neck 60 to allow for attachment of a cap (not shown) to the refill 50 and an annular projection 68 extends outwardly from the neck 60 below the threading 66. The annular projection 68 may optionally be discrete projections, one or more grooves, or any other feature that interacts with the latches 28A, 28B to retain the refill 50 within the dispenser 20, as will be discussed in greater detail hereinafter.

The volatile material 54 disposed in the container 52 may be of any type of volatile material adapted to be dispensed into the air. For example, the volatile material 54 may be a cleaner, an insecticide, an insect repellant, an insect attractant, a mold or mildew inhibitor, a fragrance, a disinfectant, an air purifier, an aromatherapy scent, an antiseptic, a positive fragrancing volatile material, an air-freshener, a deodorizer, or the like, and combinations thereof. Additives may be included in the volatile material, such as, for example, fragrances, and/or preservatives.

Referring still to FIG. 3, the plug assembly 64 is generally rigid and includes an outer annular ring 80 that is attached to the neck 60 of the container 52 and a generally circular portion 82 with a circular cutout 84 in a central portion thereof, wherein the circular portion 82 extends inwardly from a top portion 83 of the annular ring 80. The plug assembly 64 further includes a cylindrical portion 85 extending upwardly from the circular portion 82 at the circular cutout 84. Any known mechanism for retaining the plug assembly 64 may be utilized, including but not limited to, projections and grooves, adhesive, an interference fit, a snap fit, a bayonet fit, and the like.

The wick 58 is elongate such that a bottom portion 100 of the wick 58 extends into contact with and absorbs the volatile material 54 disposed within the container 52. A top portion 102 of the wick 58 extending upwardly from the plug assembly 64 is exposed to the ambient environment. The wick 58 is depicted as a substantially uniform cylinder with a substantially constant diameter throughout the entire length thereof. However, the wick 58 may be other shapes and sizes as known in the art. The wick 58 may be made of one material or alternatively may be made of multiple materials, for example, the bottom portion 100 of the wick 58 may be made of a first material and the top portion 102 of the wick 58 may be made of a second material. Materials suitable for the wick 58 generally include polymeric or porous materials and/or any other material known in the art that are suitable for wicking.

As seen in FIG. 3, the refill 50 may be inserted into and used with the dispenser 20 by first inserting the wick 58 thereof into the channel 26 of the dispenser 20. Once the wick 58 is aligned within the channel 26, the refill 50 is moved upwardly into the dispenser 20 and the latches 28A, 28B move outwardly to accommodate the annular projection 68. As the annular projection 68 passes the latches 28A, 28B, the latches 28A, 28B return to their original position and interfere with the projection 68 to functionally retain the refill 50 within the dispenser 20. The refill 50 may be removed from the dispenser 20 by exerting a downward force on the refill 50, which, in turn, exerts downward and outward pressure from the projection 68 on the latches 28A, 28B, thereby forcing the latches 28A, 28B outwardly such that the refill 50 may be removed therefrom. Once the refill 50 is secured in the dispenser 20, at least a portion of the wick 58 is preferably exposed to allow heat from the heater (if used) in the dispenser 20 to evaporate the volatile material 54 that is moved to the top portion 102 of the wick 58 through capillary action.

The refill 50 in FIG. 3 is a compatible refill for the dispenser 20 because the refill 50 can be attached to and retained within the housing 22 by the latches 28A, 28B with the refill 50 disposed within the cavity 24 and the wick 58 may be inserted far enough into the channel 26 to allow for attachment and retention by the latches 28A, 28B. More particularly, the obstruction 30 is positioned within the channel 26 so that there is sufficient clearance for at least the top portion 102 wick 58 to be received into the channel 26 when the latches 28A, 28B grasp and retain the refill 50 by the annular projection 68. The arrangement of the refill 50 within the dispenser 20 is dependent, in part, upon the distance X between the obstruction 30 and top portions of the latches 28A, 28B corresponding to the distance Y between the top of the wick 58 and the annular projection 68. Specifically, for a compatible refill, the distance X should be greater than or equal to the distance Y. In one example, the distance between a very top of the wick 58 and the annular projection 68 is about 3.5 cm and the distance between the obstruction 30 and the latches 28A, 28B is greater than about 3.5 cm.

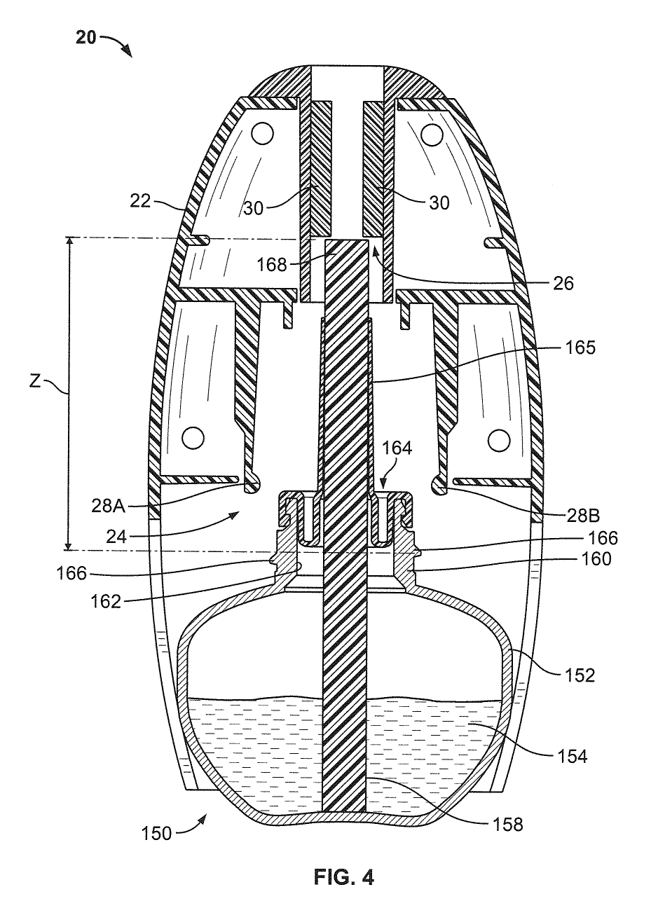

FIG. 4 illustrates an incompatible refill 150 that is similar to the refill 50. In particular, the refill 150 includes a bottle or container 152 with a volatile material 154 disposed therein, wherein a wick 158 is in contact with the volatile material 154 and extends out of the container 152. The container 152 includes a neck 160 that extends from an upper portion of the container 152 and includes an opening 162 that allows access to the volatile material 154. A plug assembly 164 is disposed within the opening 162 of the neck 160 of the container 152 to hold the wick 158 within the container 152. The plug assembly 164 has an optional sheath 165 extending upwardly therefrom and surrounding at least a portion of the wick 158. Although the sheath 165 is shown as ending prior to a top of the wick 158, the sheath 165 could extend to the top of the wick 158 or extend beyond a top of the wick 158. Annular threading 166 is disposed around the neck 160 to allow for attachment of a cap (not shown) to the refill 150. The plug assembly 164 of the incompatible refill 150 is shown as being different from the plug assembly 164 of the compatible refill 50, but may alternatively be the same. The refills 50, 150 may have different shapes and sizes, but the most notable difference is that the incompatible refill 150 has a longer wick 158 that extends from the neck 160 of the container 152 by a greater distance than the wick 58 extends out of the refill 50. This being the case, the distance Z between a very top 168 of the wick 158 and the threading 166, which would be intended to retain the refill 150 within the dispenser 20, is greater than the distance Y (FIG. 3) between the very top of the wick 58 and the annular projection 68 of the compatible refill 50 and also greater than the distance X (FIG. 3) between the obstruction 30 and the top portions of the latches 28A, 28B. Consequently, when the wick 158 is inserted into the channel 26, the obstruction 30 prevents the wick 158 from being inserted far enough into the channel 26 such that the latches 28A, 28B grasp any portion of the threading 166 on the refill 150 and retain the refill 150 within the dispenser 20. Optionally, if the sheath 165 is present in the refill 150, the sheath 165, rather than the wick 158, may prevent insertion of the sheath 165 and wick 158 far enough into the channel 26 such that the latches 28A, 28B grasp any portion of the threading 166 to retain the refill 150 within the dispenser 20.

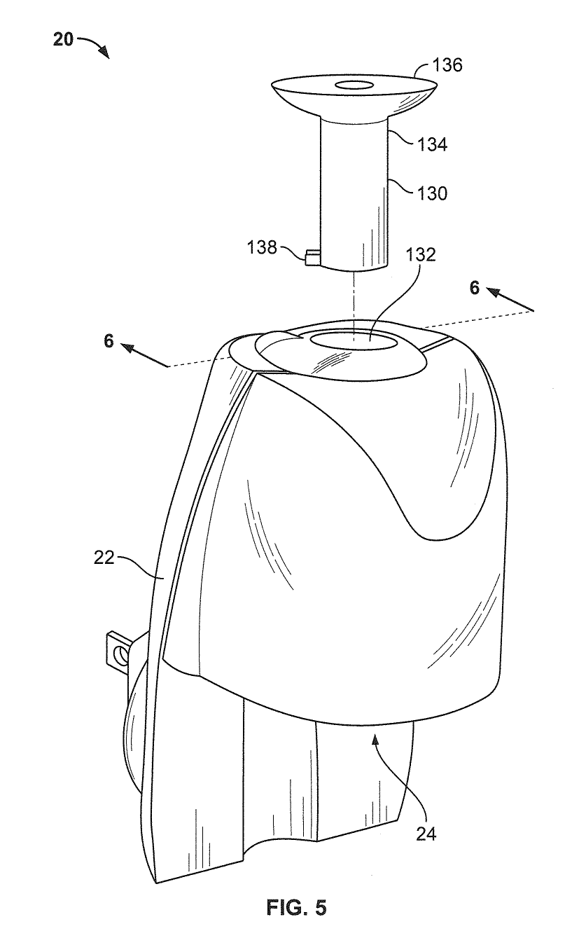

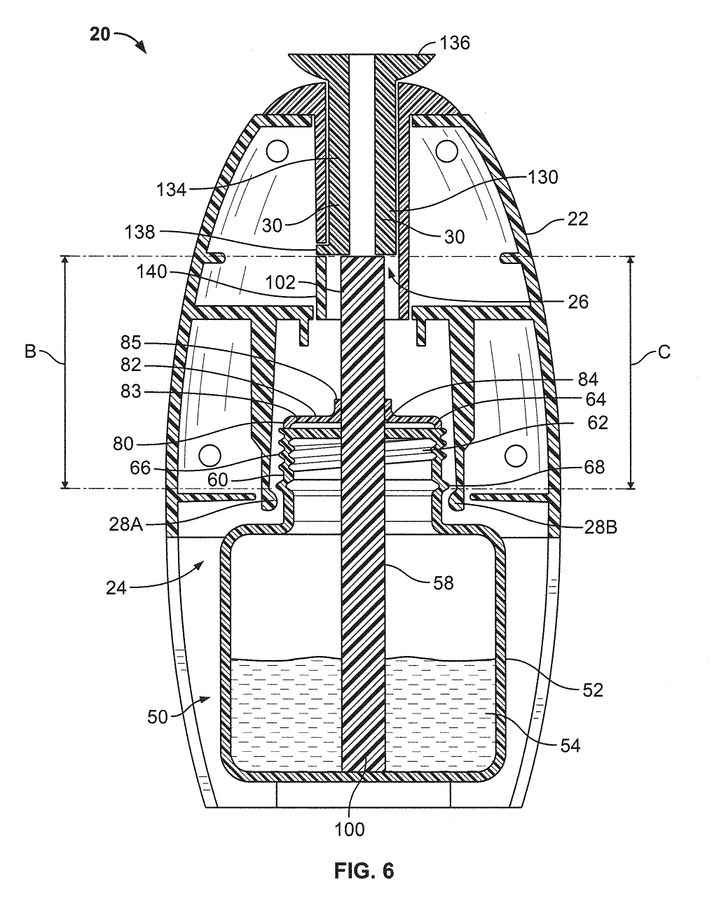

FIG. 5 illustrates another embodiment of the dispenser 20 that is identical to the dispenser 20 of FIG. 1 but includes a different embodiment of the obstruction 30. In FIG. 5, the obstruction 30 is an adapter 130 that is secured within the channel 26. The adapter 130 can be a decorative element that is inserted into an aperture 132 in a top portion of the housing 22 and secured in the channel 26. In one embodiment, as seen in FIGS. 5 and 6, the adapter 130 includes a cylindrical member 134 that is preferably hollow through a central portion thereof so as to not prevent emission of volatile material from the dispenser 20 and a top member 136 that extends from a top portion of the cylindrical member 134. The top member 136 may have decorative features, indicia, or colors thereon. A discrete projection 138 extends outwardly from a portion of the cylindrical member 134 and is adapted to be inserted into an aperture 140 (FIG. 6) formed by walls forming the channel 26. The projection 138 may be separated from a remainder of the cylindrical member 134 by slots to allow the portion of the cylindrical member 134 having the projection 138 to deform inwardly as the adapter 130 is inserted into the channel 26 and flex back to its normal position (as seen in FIG. 6) as the projection 138 reaches the aperture 140. Alternatively, the adapter may be secured within the channel 26 or to the housing 22 by any means know in the art, e.g., an interference fit, adhesive, a snap fit, a bayonet fit, and the like.

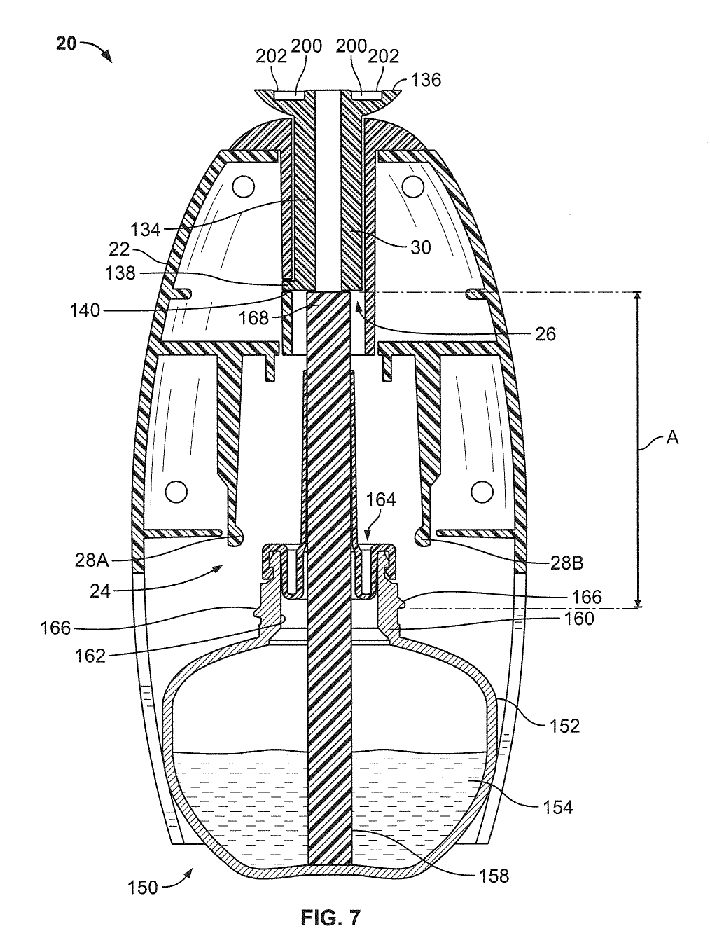

FIG. 6, like FIG. 3, illustrates a compatible refill 50 that can be attached to the housing 22 by the latches 28A, 28B with the refill 50 disposed within the cavity and the wick 58 inserted far enough into the channel 26 such that the latches 28A, 28B interact with and retain the refill 50 by the projection 68. FIG. 7, like FIG. 4, illustrates an incompatible refill 150 where the adapter 130 prevents the wick 158 or a sheath surrounding the wick 158, as discussed in greater detail hereinabove, of the refill 150 from being inserted far enough into the channel 26 such that the threading 166 is not retained in the dispenser 20 by the latches 28A, 28B, thereby preventing use of the refill 150 in the dispenser 20.

As with the embodiment of FIGS. 1-4, the refills 50, 150 may have different shapes and sizes, but the most notable difference is that the incompatible refill 150 has a longer wick 158 that extends from the neck 160 of the container 152 by a greater distance than the wick 58 extends out of the refill 50. This being the case, the distance A (FIG. 7) between a very top 168 of the wick 158 and the threading 166, which would be intended to retain the refill 150 within the dispenser 20, is greater than the distance B (FIG. 6) between the very top of the wick 58 and the annular projection 68 of the compatible refill 50 and also greater than the distance C (FIG. 6) between a bottom portion of the adapter 130 and the top portions of the latches 28A, 28B. Consequently, when the wick 158 is inserted into the channel 26, the obstruction 30 prevents the wick from being inserted far enough into the channel such that the latches 28A, 28B grasp any portion of the threading 166 on the refill 150 and retain the refill 150 within the dispenser 20.

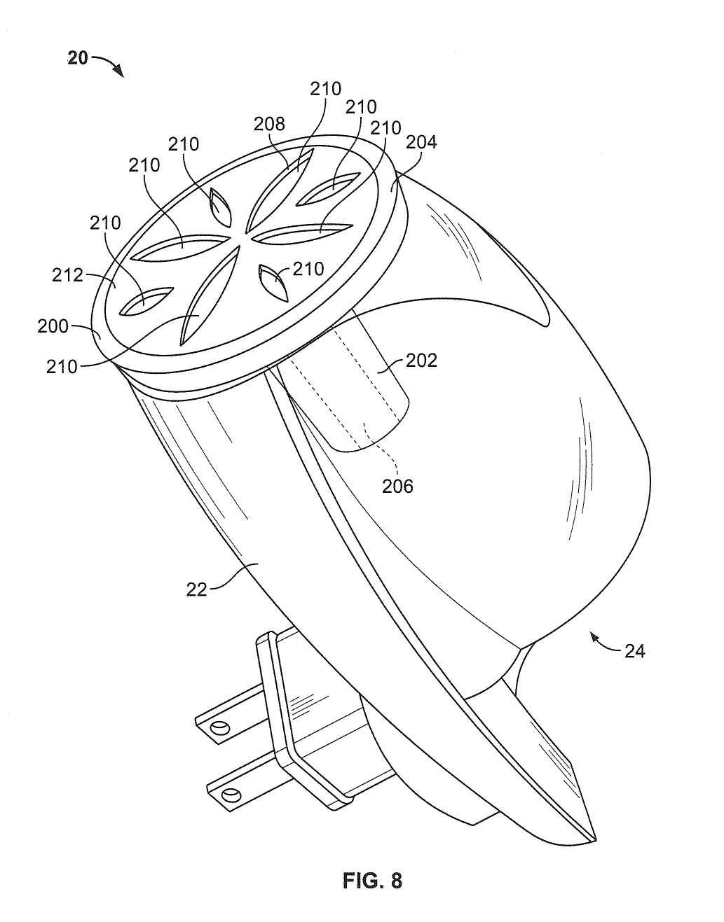

FIGS. 8-10 depict further embodiments of the dispenser 20 that are identical to the dispenser 20 of FIG. 5, but include different embodiments of obstructions 30 in the form of adapters 130. Specifically, FIG. 8 depicts an adapter 200 having a hollow cylindrical member 202 and a top member 204 that extends from a top portion of the cylindrical member 202, similar to the adapter 130 of FIG. 5. In the embodiment of FIG. 8, rather than having a channel 206 through the entirety of the adapter 200, the channel 206 ends at a chamber 208 in the top member 204. The top member 204 includes a plurality of apertures 210 in a top surface 212 thereof to emit volatile material therefrom. The adapter 200 may also include a set of louvers or other adjustment mechanism (not shown) that may be controlled by a user to adjust a size of the apertures 210 to emit more or less volatile material therefrom.

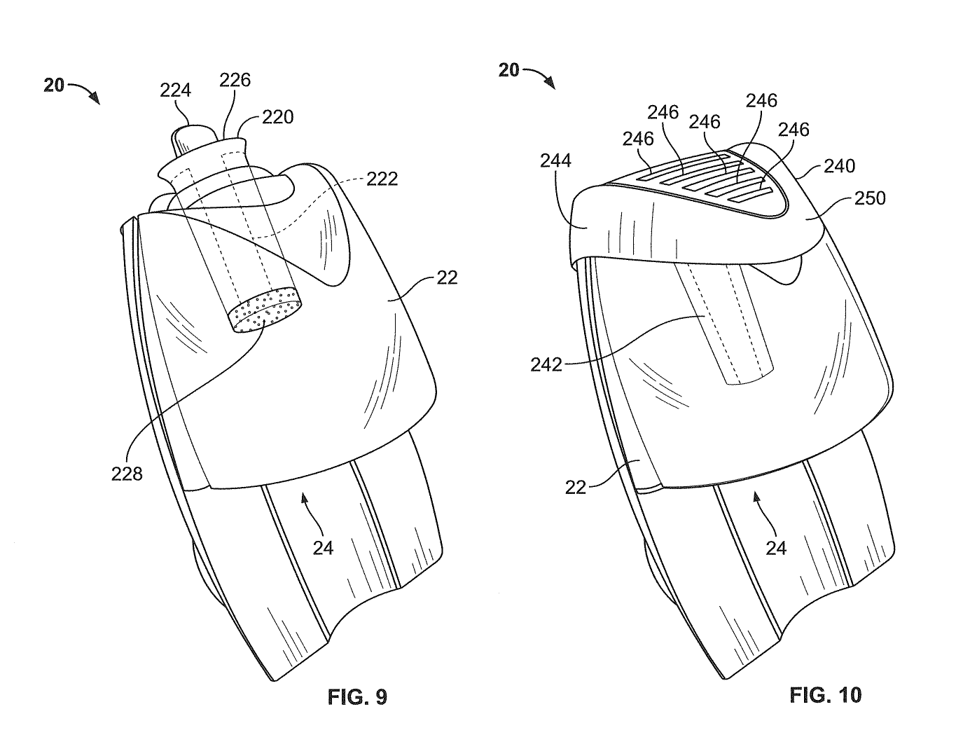

Referring to FIG. 9, the dispenser 20 includes an adapter 220 having a hollow cylindrical member 222 and an adjustment mechanism 224 disposed in a top portion 226 of the cylindrical member 222 to allow a user to adjust the amount of volatile material emitted from the dispenser 20. The adapter 220 may also include a porous member 228 at a bottom portion 230 thereof, wherein the porous member 228 is impregnated with a volatile material, either the same or different from that in a refill utilized with the dispenser 20.

The dispenser of FIG. 10 includes an adapter 240 having a hollow cylindrical member 242 and a top member 244 that extends from a top portion of the cylindrical member 242. The top member includes a plurality of vents 246 therethrough and includes an optional shroud 248 that attaches to and covers a top portion 250 of the dispenser 20. A set of louvers or other adjustment mechanism (not shown) may be utilized with the vents 246 to allow a user to adjust a size of the vents 246 to emit more or less volatile material therefrom.

The adapters 200, 220, 240 of FIGS. 8-10 may be inserted into and attached to the dispenser 20 in any manner described herein. Further, the adapters 200, 220, 240 work in the same manner as the obstruction 30 and adapter 130 of FIGS. 1 and 5 to prevent insertion of a wick or sheath of an incompatible refill into the dispenser 20, yet allow for attachment of a compatible refill.

The obstruction 30 of FIGS. 2-4, the adapter 130 of FIGS. 5-7, or the adapters 200, 220, 240 of FIGS. 8-10, may incorporate one or more volatile materials therein. Referring to FIG. 7, a modified adapter 130 is depicted in which one or more cavities 200 are formed with a gel in the form of a fragrance therein. The cavities 20 may each include a semi-permeable membrane 202 disposed over a top portion thereof to allow controlled emission of the fragranced gel therefrom. The volatile material may be passively emitted, sprayed, or otherwise emitted in a fashion know in the art to provide a burst or increase in volatile material. The volatile material may be the same or different from the volatile material contained in the refill. Further, the volatile material may be impregnated within, applied to, disposed within a cavity or aperture, attached to, or otherwise contained within the obstruction 30 or adapter 130 and the volatile material may be in any form, for example, liquid, aerosol, gel, solid, and the like.

The dispensers employing the refills described herein may comprise a variety of forms as known in the art. The dispensers may optionally use electric power in the form of batteries or an electrical plug during the operation thereof. The dispensers also optionally may include one or more of a heater, a fan, and/or other components disposed in a housing that help facilitate the release of the volatile active. The dispensers may further include one or more openings in the housing to allow the volatile material to be dispensed from the housing to the surrounding environment. In place of the latches 28A, 28B, the housing 22 may include a variety of internal implements or couplings to help secure the various refill bottles disclosed herein, such as, for example, snaps, ridges, undercuts, lips, notches, projections, grooves, interference fit, threading, and/or any other attachment methods known in the art. The dispenser may optionally include one or more refills and may operate using a variety of timing sequences as known in the art.

Any of the embodiments described herein may be modified to include any of the structures or methodologies disclosed in connection with other embodiments.

### INDUSTRIAL APPLICABILITY

The present invention provides a volatile material dispenser that is adapted for use with only compatible volatile material refills. In particular, an obstruction within a channel of the dispenser prevents incompatible refills from being coupled to or retained within the dispenser.

Numerous modifications to the present disclosure will be apparent to those skilled in the art in view of the foregoing description. Accordingly, this description is to be construed as illustrative only and is presented to enable those skilled in the art to make and use the disclosure and to teach the best mode of carrying out same. The exclusive rights to all modifications that come within the scope of the appended claims are reserved.

I claim:

1. A volatile material dispenser, comprising:
a housing;
means for retaining a refill containing a volatile material to the housing;
a channel disposed within the housing for receiving a wick disposed within the refill; and
a member disposed within the channel;
wherein a first refill having a first container for holding a volatile material and having a first wick in contact with the volatile material and extending out of the first container is capable of being attached to the housing by inserting the first wick into the channel until the means for retaining retains a portion of the first refill; and
wherein a second refill having a second container for holding a volatile material, a second wick in contact with the volatile material and extending out of the second container, and an optional sheath disposed around the second wick is prevented from being attached to the housing by the member, which prevents insertion of the second wick or the sheath far enough into the channel for the refill to be retained by the means for retaining, thereby preventing retention of a portion of the second refill by the means for retaining.

2. The volatile material dispenser of claim 1, wherein the member is a projection that extends from a wall forming the channel.

3. The volatile material dispenser of claim 1, wherein the member is an adapter secured within the channel.

4. The volatile material dispenser of claim 3, wherein the adapter is secured within the channel by an interference fit.

5. The volatile material dispenser of claim 3, wherein the adapter is secured within the channel by a bayonet-type connection.

6. The volatile material dispenser of claim 1, wherein the first wick extends out of the first bottle by a first distance and the second wick extends out of the second bottle by a second distance, and wherein the first distance is less than the second distance.

7. The volatile material dispenser of claim 1, wherein the means for retaining a bottle includes one or more resilient latches that clip under one or more protrusions extending from the first bottle.

8. The volatile material dispenser of claim 7, wherein a distance between a bottom of the member and the one or more latches is equal to or greater than a distance between a top of the first wick and the one or more protrusions of the first bottle.

9. The volatile material dispenser of claim 8, wherein a distance between a bottom of the member and the one or more latches is less than a distance between a top of the second wick and one or more protrusions extending outwardly from the second bottle.

10. The volatile material dispenser of claim 8, wherein the distance between the top of the first wick and the one or more protrusions of the first bottle is about 3.5 cm.

11. A method of allowing only compatible refills to be retained by a volatile material dispenser, the method comprising the steps of:
providing a volatile material dispenser that includes a housing, a channel disposed within the housing, a member disposed within the channel, and a coupling for retaining a refill;
providing a compatible refill that includes a container for holding a volatile material and a wick extending from the container; and
inserting the refill into the housing so that the wick extends into the channel and the coupling retains a portion of the refill,
wherein the member prevents a wick or a sheath surrounding the wick of an incompatible refill from being inserted far enough into the channel for the coupling to retain a portion of the incompatible refill, thereby preventing attachment of the incompatible refill to the dispenser.

12. The method of claim 11, wherein the member is a projection that extends from a wall forming the channel.

13. The method of claim 11, wherein the member is an adapter secured within the channel.

14. The method of claim 11, wherein the coupling includes one or more latches that clip under one or more protrusions extending from the bottle.

15. The method of claim 14, wherein a distance between a bottom of the member and the one or more latches is equal to or greater than a distance between a top of the first wick and the one or more protrusions of the bottle.

16. The method of claim 11, further including the step of providing a volatile material within the member to enhance the emission of volatile materials from the dispenser.

17. A volatile material dispenser, comprising:
a housing adapted to retain a compatible refill, wherein the refill includes a container for holding a volatile material and a wick extending from the container;
a channel disposed within the housing for receiving the wick;
a coupling disposed within the housing for attaching the refill to the housing; and
an obstruction disposed within the channel that allows the wick of the compatible refill to be inserted far enough within the channel such that the compatible refill can be retained by the coupling, wherein the obstruction prevents a wick or a sheath surrounding the wick of an incompatible refill from being inserted far enough within the channel to retain the incompatible refill by means of the coupling.

18. The volatile material dispenser of claim 17, further including the step of providing a volatile material within the member to enhance the emission of volatile materials from the dispenser.

19. The volatile material dispenser of claim 17, wherein the obstruction is a decorative adapter that is inserted into a top of the housing and secured within the channel.

20. The volatile material dispenser of claim 17, wherein the coupling includes at least one resilient latch.
